(19) United States
(12) Patent Application Publication
(10) Pub. No.: US 2018/0146936 A1
(43) Pub. Date: May 31, 2018

(54) SYSTEM FOR THE LOCALIZED MEASUREMENT OF RADIOTRACER IN THE BODY

(71) Applicant: Lucerno Dynamics, LLC, Cary, NC (US)

(72) Inventors: Joshua G. Knowland, Cary, NC (US); Ronald K. Lattanze, Morrisville, NC (US); Jesse Kingg, Cary, NC (US); Paul Mozley, Collegeville, PA (US); William Gorge, Carmel, IN (US); Charles W. Scarantino, Raleigh, NC (US); Steven Perrin, Raleigh, NC (US)

(73) Assignee: Lucerno Dynamics, LLC

(21) Appl. No.: 15/824,624

(22) Filed: Nov. 28, 2017

Related U.S. Application Data
(60) Provisional application No. 62/426,918, filed on Nov. 28, 2016.

(51) Int. Cl. A61B 6/00; A61M 5/00; G01T 1/161; G01T 1/167

(52) U.S. Cl. CPC A61B 6/4057; A61M 5/007; A61B 2090/3614; G01T 1/167; G01T 1/1617

(57) ABSTRACT

Various embodiments of a device for in-vivo measurements radiopharmaceuticals used for diagnosis and radiotherapy is presented. In some embodiments, the present disclosure relates to a scintillation device having a cannula that may include scintillation material and a delivery lumen, wherein the device may be used to both deliver material to the patient (e.g., deliver radiotracers used in radiopharmaceuticals) and measure levels of radioactive material in, for example, the patient's blood both during and after administration of the radioactive material. In some embodiments, particles emitted by the radioactive material interact with the scintillation material, resulting in the release of light that may be transmitted, via the scintillation material and/or fiber optic material, to one or more optical detectors or processors for processing. In some embodiments, particle absorbing materials may be used to reduce the effective measurement volume thereby measure only particles emitted from within a blood vessel of interest.

### PRIORITY

This patent application claims the priority of U.S. provisional patent application No. 62/426,918 titled System for the Localized Measurement of Radiotracer in the Body, filed on Nov. 28, 2016, the entirety of which is incorporated herein by reference.

### TECHNICAL FIELD

The present disclosure is related generally to a device and/or system for the localized measurement of radiotracers in fluids or tissue. More specifically, the present disclosure relates to (1) various embodiments of devices configured for, among other things, the in-vivo measurement of radioactive material (RAM) in the tubing used to administer radiopharmaceuticals for diagnosis or radiotherapy; RAM in blood within various types of blood vessels; the in-vivo measurement of RAM in various tissues accessed through angiography, such as the liver, heart and brain; and the ex-vivo measurement of RAM in other biological compartments; and (2) analogous measurements of RAM in non-biological fluids flowing through fabricated industrial conduits.

### BACKGROUND

The present disclosure offers certain improvements in a variety of different contexts. For example, many physiological studies, including those in which the outcome measure is analogous to a rate of metabolism of a biological substance, or the concentration of a target protein on cell surfaces, or the activity of enzymes in tissues, and the like, use compartmental modeling to solve the rate equations which requires measuring the change in the concentration of a radiotracer available in the blood supply over time. To obtain this “arterial input function” (AIF), multiple samples of the fluid of interest, such as, for example, blood, are aspirated from the conduit, for example a blood vessel (including arteries), and analyzed in vitro. Repeatedly drawing blood from a cannulated artery is currently accepted as the most rigorous way to characterize the AIF in medical research. While many investigators have shown that it is feasible to perform serial arterial punctures within some subjects, tolerance for such research procedures can vary in populations that have various types of complicating medical issues.

Furthermore, repeated punctures may slow subject accrual or contribute to subject dropout rates in longitudinal studies. Risks may also increase in aging populations who require medications for co-morbid conditions that have an effect on the blood clotting cascade. Even when the procedure goes relatively well from the perspective of the patient, many sources of variance enter the system and adversely affect the precision of measurement. Confounds may include challenges in aspirating standard amounts of blood that have not been diluted with the saline solutions that keep catheters from clotting shut, estimating the mean time of aspiration for a process that takes time to complete, problems synchronizing clocks between the various measurement devices, and many others.

In other contexts, Angiography and the selective intra-arterial administration of RAM for the treatment of cancer is a growing field. Evidence continues to mount that loco-regional radiotherapy reduces morbidity and prolongs survival in patients with a variety of cancers. At this time, delivery of the RAM from the injection vial to the intended site must be inferred. While it is possible to measure decreases in radioactivity in the injection vial with an external measuring device based on, for example, gas ionization chamber technology, and it is possible to administer radiopaque contrast to follow the flow of fluids through the catheters and selected arteries, it is not possible to measure RAM in the selected arterial system in real time while the procedure is in progress. Additionally, reliance on radiopaque contrast material to detect backflow to tissues that should not be treated increases the risks of radiation-induced injuries to bystander tissues.

Accordingly, there remains a need to overcome the challenges associated with measuring the levels or concentrations of radiotracer available in a vessel or other area in the body over a certain period of time.

### SUMMARY

A novel solution to certain of the challenges outlined above may include insertion of a device having a radiotracer detector into fluid carrying vessel (e.g., a blood vessel, pipe, etc.). In so doing, AIF measurements could be obtained in vivo (or in situ). Such a detector may be advantageously integrated with an intravenous, intra-arterial, or any other intra-luminal catheter (or other intra-vessel device) which may be used to inject the radiotracer, thereby reducing the number of points at which access to the blood supply (or other fluid) are needed. Such in vivo (or in situ) measurements allow the procedures to be performed more consistently than existing techniques because, for example, aspirating fluid from the same catheter that was used for injection can cause backflow of stagnant radioactivity pooling in the circulation.

Backflow of stagnant RAM can artifactually elevate the estimate of the average concentration of radioactivity in the blood. Conversely, the backflow of normal saline that may be steadily dripped at a rate to keep the vein open can dilute the aspirated blood (or other fluid) and lead to artifactually decreased concentrations of radioactivity. By using devices such as those described herein, aspirating fluid is substantially prevented from reversing the direction of flow. This obviates the need to insert separate catheters for injection and aspiration, thereby improving the experience for both subject and practitioner.

In certain other embodiments, such as for example, industrial settings, retrofitting aging or constructing new conduits with in-situ embodiments of this present invention would allow for remote continuous monitoring for RAM. Advantages can include, among other things, reductions in the human and economic costs of manual interventions.

According to some embodiments of the present disclosure, a scintillation device for the localized measurement of radiotracers in the body is presented. The device may include, among other things, a cannula having scintillation material and one or more delivery lumens, wherein the scintillation material emits light when impacted with certain particles that may be emitted from, for example, a radioactive material. In some embodiments, the cannula may be sized to deliver the delivery lumen and the scintillation material to a position inside a blood vessel of interest. In some embodiments, the device may further include an optical connector or optical detector that may receive at least a portion of the light emitted from the scintillation material.

The device may also include, in some embodiments, fiber optic material for transmitting light emitted from the scintillation material to the optical connector or detector. The scintillation material may also be shaped to desirably focus the light emitted by the scintillation material advantageously towards the fiber optic material to facilitate better transmission of light to the optical detector or connector. In some embodiments, a lens may also be incorporated. Upon receipt by the optical connector or optical detector, the light may be converted to electrical signals for processing. The cannula may include, in some embodiments, needle material as used, for example, in hypodermic needles, or may alternatively include any other material, including biocompatible plastics and the like that may be used in catheters, etc.

In some embodiments, in may be advantageous to include one or more wings that may be used for substantially centering the device within the blood vessel of interest. The one or more wings may, in some embodiments, be operatively movable from a first retracted position to a second extended position. Accordingly, in may be possible to deliver the scintillation device to the blood vessel of interest with the one or more wings in the first retracted position (thereby minimizing the overall diameter of the device during insertion), and then extend the one or more wings to the second extended position once inside the blood vessel to, for example, position the device substantially within the center of the blood vessel. The one or more wings may subsequently be retracted to the first retracted position for removal of the device from the blood vessel of interest.

In some embodiments, in may be advantageous to limit the effective measuring volume of the scintillation device to an area that would fall within the blood vessel of interest for a plurality of patients having blood vessels of different diameters (for example, from between about 5 mm to about 10 mm, or from about 1 mm to about 20 mm, or more). In various embodiments, the scintillation device may include one or more layers of particle absorption material configured to effectively block particles below a certain energy threshold (e.g., particles emitted from outside a desired measurement volume). For example, the particle absorption material may include an energy blocking threshold corresponding to its absorption of energy from the particles emitted from the radioactive material, and further wherein the particle absorption material may include a thickness configured to effectively block particles having an energy at the particle absorption material below a desired threshold. The particle absorption material may include one or more of PEEK, gold, or various other materials capable of absorbing certain amounts of energy from the particles.

In some embodiments of the present disclosure, the scintillation device may include a particle absorption material positioned between the delivery lumen and the scintillation material having a first energy blocking threshold, and particle absorption material positioned elsewhere within the device that includes a second energy blocking threshold, or in some embodiments, two or more energy absorption thresholds.

In various other embodiments of the present disclosure, a scintillation device for localized measurement of radiotracers in a blood vessel is presented that includes a delivery lumen and scintillation material, wherein the delivery lumen is coupled to a delivery hub, and the scintillation material is optically coupled to a light detector. The light detector may, in some embodiments, be housed within the device, and may include a signal port for transmitting a signal to an external reader. In some embodiments, the scintillation material axially surrounds the delivery lumen, and the device may also include a first area of particle absorption material between the delivery lumen and the scintillation material having a first energy blocking threshold. The device may further include a second area of particle absorption material axially surrounding the scintillation material having a second energy blocking threshold (that may or may not be substantially equal to the first energy blocking threshold).

### BRIEF DESCRIPTION OF THE DRAWINGS

Having thus described the presently disclosed subject matter in general terms, reference will now be made to the accompanying Drawings, which are not necessarily drawn to scale, and wherein:

FIG. 1 illustrates a side view cross section of an exemplary closed end scintillator needle according to one aspect of the present disclosure.

FIG. 2 illustrates a side view cross section of an exemplary open end scintillator needle according to another aspect of the present disclosure.

FIG. 3 illustrates a side view cross section of an exemplary blunt end scintillator probe according to another aspect of the present disclosure.

FIG. 4 illustrates a side view cross section of an exemplary scintillator cannula according to another aspect of the present disclosure.

FIG. 5A illustrates a cross sectional view along a longitudinal axis of an exemplary scintillator cannula as shown, for example, in FIG. 4, according to another aspect of the present disclosure.

FIG. 5B illustrates a cross sectional view along a longitudinal axis of an alternative exemplary scintillator cannula similar to the cannula shown in FIG. 4, but having more than one delivery lumen, according to another aspect of the present disclosure.

FIG. 6 illustrates a side view cross section of yet another blunt end scintillator probe according to other aspects of the present disclosure.

FIG. 7 illustrates a side view cross section of yet another blunt end scintillator probe according to other aspects of the present disclosure.

FIG. 8 illustrates cross sectional view along a longitudinal axis of an alternative exemplary embodiment of the present disclosure, wherein the probe taught for example in FIG. 7 is deployed inside, for example, a catheter.

FIG. 9 illustrates a side view cross section of an exemplary embodiment of the present disclosure employing a lens for, among other things, focusing scintillation light.

FIG. 10 illustrates a side view cross section of yet another embodiment of the present disclosure wherein the scintillation material may be shaped for purposes of, for example, focusing scintillation light.

FIG. 11 illustrates a side view cross section of an alternative embodiment of the present disclosure that includes an optical detector coupled to the scintillation material, along with associated electric cabling that may be utilized to transmit an electric signal from the optical detector for processing.

FIG. 12 illustrates a variation of the embodiment shown in FIG. 11 that includes a relatively small portion of fiber optic material between the scintillation material and the optical detector.

FIG. 13 illustrates a side view cross section of yet another embodiment of the present disclosure that includes scintillation material adjacent a length of a delivery lumen and terminating within material surrounding a delivery hub.

FIG. 14 illustrates a side view cross section of another embodiment similar to that illustrated in FIG. 13, but wherein the delivery lumen itself includes or is constructed from scintillation material.

FIG. 15A illustrates a side view cross section of another embodiment similar to that illustrated in FIG. 13, but wherein one or more light detectors are mounted at a longitudinal end of the scintillation material/delivery lumen.

FIG. 15B illustrates a longitudinal side view cross section of the device illustrated in FIG. 15A.

FIG. 16 illustrates a side view cross section of another embodiment of the present disclosure wherein one or more light detectors are positioned radially on the scintillation material.

FIG. 17 illustrates a variation of device illustrated in FIG. 16 wherein the scintillation material may include a redirecting surface to redirect light traveling along the longitudinal axis of the scintillation material to a more radial direction.

FIG. 18 illustrates a side view cross section of another embodiment of the present disclosure wherein four light detectors are positioned radially around a circular scintillation material to capture incident light.

FIG. 19 illustrates a longitudinal cross section of the embodiment shown in FIG. 18.

FIG. 20A and FIG. 20B illustrate side and perspective views, respectively, of an exemplary mechanism for substantially centering the presently closed devices in a blood vessel of interest, in a first retracted position, according to one embodiment.

FIG. 21A and FIG. 21B illustrate side and perspective views, respectively of the mechanism in FIG. 20A and FIG. 20B, but in a second extended position.

### DETAILED DESCRIPTION

The presently disclosed subject matter now will be described more fully hereinafter with reference to the accompanying Drawings, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

In some embodiments, known scintillation materials, such as for example, organic, inorganic, and/or plastic scintillation materials, may be configured to be inserted into a fluid carrying vessel (e.g., a blood vessel) for use in measuring levels of RAM in the fluid carried within the vessel. Such scintillation materials are known to interact with certain RAM and generate light in response. Such light can then be detected using various detectors and used to determine the presence of, and if applicable the level of, RAM in the fluid. Such scintillation materials may also be used to measure the presence of, and if applicable the level of, RAM in tissues in the body, or other materials. Plastic-based scintillation fibers are commercially available in the art. Such plastic-based scintillation fibers typically consist of scintillation material incorporated into a plastic resin which is then extruded into thin fibers. Commonly available sizes include diameters from 0.25 mm to 5 mm. Of course, any suitable scintillation material may be employed depending on the application. Suitable scintillation materials, and systems and methods for externally detecting, measuring, and analyzing signals to determine the levels of RAM present in an area of interest are known by those having skill in the art, such as, for example, the systems and methods taught in U.S. Pat. No. 9,002,438 and/or U.S. patent application Ser. No. 14/678,550, both of which are incorporated herein by reference in their entirety.

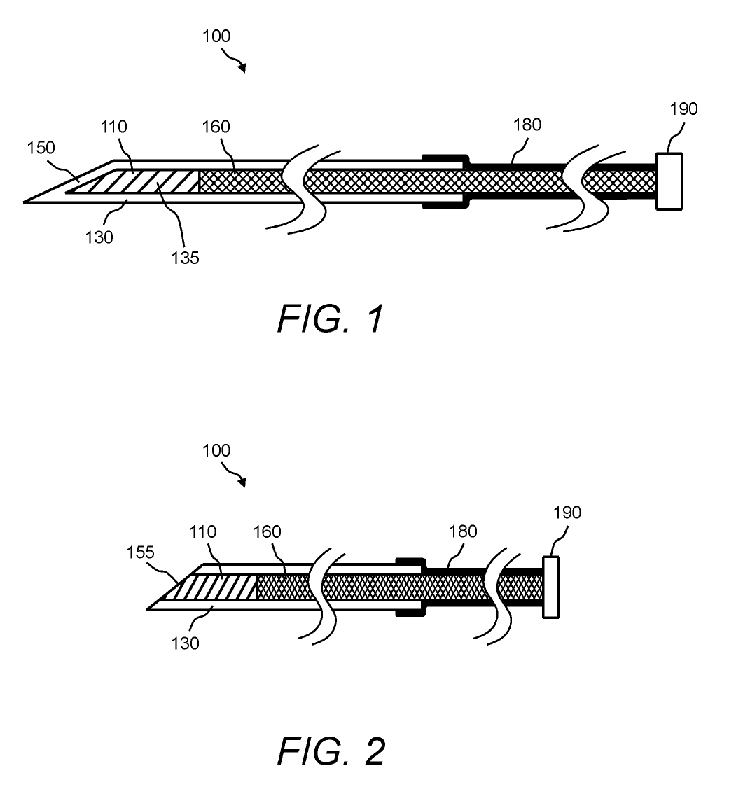

Referring now to FIG. 1, an exemplary embodiment of a scintillator probe 100 according to some embodiments of the present disclosure is presented. The exemplary scintillator probe 100 presented in FIG. 1 may include scintillation material 110 within a needle 130 formed from any suitable needle material. In some embodiments, the scintillation material 110 may be positioned within a hollow core 135 of the needle 130. In some embodiments, the needle 130 may have a closed end 150, such that scintillation material 110 may be substantially enclosed within needle 130. The hollow core 135 of the needle 130 may further include fiber optic material 160 capable of propagating or transmitting light emitting from the scintillation material 110 to an optical connector 190. Various embodiments of the present disclosure could use the optical connector 190 to couple the scintillator probe 100 to a separate optical sensor (not pictured) for measurement of the light signal. Any known or later discovered fiber optic material 160 may be utilized based on the application for the device. In some embodiments, light shielding material 180 may be utilized to, among other things, prevent unwanted external light from being transmitted to the scintillation material 110, fiber optic material 160, and/or the optical connector 190. Any appropriate light shielding material 180 may be utilized as needed to prevent, for example, contamination from unwanted light. It will be understood that the fiber optic material 160 may be used to propagate or transmit the light signal generated in the scintillation material 110 to optical connector 190 that may be located substantially near or substantially far away from the scintillation material 110 (e.g., outside of the body).

For example, various embodiments of the present disclosure could make use of opaque light shielding materials 180 that are known in the art including, among others, metals, plastics, coatings, sealants, etc. Additionally, a light-proof coating on the outer surface of a scintillation material (e.g., scintillation material 110) or fiber optic transmission material (e.g., fiber optic material 160) can act as a reflector to maintain light within the material(s).

Various embodiments of the present disclosure could make use of fiber optic light transmission materials (e.g., fiber optic material 160) that are known in the art including, for example, glass, plastic, silicone, etc. Various fiber optic materials are commercially available from several suppliers and such materials may be optimized for various wavelengths of light, bend radii, cladding, etc. The optical light transmission materials can also include a bundle of several optical transmission fibers to increase the effective diameter of transmission fiber while maintaining flexibility, strength, and other features, as desired.

Various embodiments of the present disclosure could also include integrated features for automatically centering or positioning the presently disclosed device within the fluid-carrying vessel as needed. Such features can include fins, prongs, protrusions, whiskers, etc. Additionally, holes placed near the tip of the catheter delivery lumen could act during injection as stabilization jets to center the catheter assembly. Various exemplary embodiments of such features are discussed further hereinbelow.

Referring now to FIG. 2, an alternative embodiment of the scintillator probe 100 depicted in FIG. 1 is presented wherein the needle 130 has an open end 155 rather than a closed end (e.g., closed end 150 in FIG. 1). The scintillator probe 100 of FIG. 2 may further include scintillation material 110, needle material 130, fiber optic material 160, light shielding material 180, and/or optical connector 190.

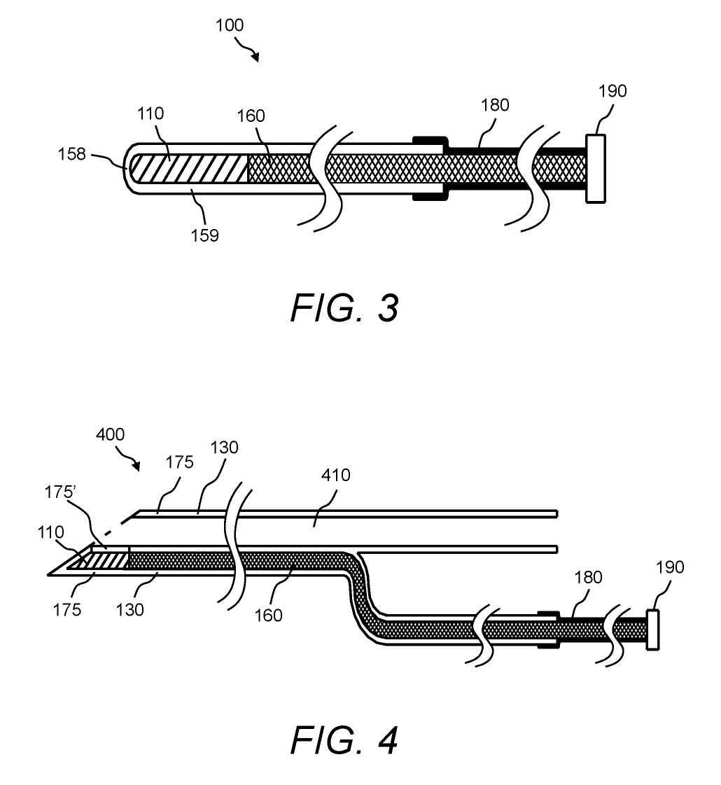

Referring now to FIG. 3, another alternative embodiment of the scintillator probe 100 is presented wherein the scintillator probe includes a blunt end 158. The probe 100 having blunt end 158 may include any suitable probe material 159, including needle material (e.g., needle material 130), plastics, metals, biocompatible variations of plastics or metals, acrylics, and/or any other suitable material known in the art. Probe 100 may also include scintillation material 110 and/or fiber optic material 160. Like the embodiments depicted in FIG. 1 and FIG. 2, the scintillator probe 100 depicted in FIG. 3 can also include light shield material 180 and one or more optical connectors 190.

Referring now to FIG. 4 an exemplary scintillator cannula 400 with integrated scintillation material 110 is presented. The scintillator cannula 400 may include a delivery lumen 410 for transmitting material into a vessel being measured (not pictured). Adjacent to or otherwise integrated with the delivery lumen 410 may also be, for example, a needle 130 within which scintillation material 110, fiber optic material 160, light shield material 180, and/or optical connector 190 may also be included.

In some embodiments of the present disclosure, it may be advantageous to limit the effective sensing range of the various scintillator probes taught herein. For example, it is often advantageous to determine the concentration of RAM in a given patient's blood stream (or other area of the body) without having to calibrate the device to the specific vessel size or area of interest in each patient. (i.e., it may be advantageous to use scintillator probes having the same specifications on a multitude of patients having, for example, blood vessels of varying sizes to take the same measurement—concentration of RAM). A difficulty, however, lies in at least the fact that a patient having a larger blood vessel will have more RAM flowing by the sensor at a given period of time relative to a patient with a smaller blood vessel, simply by virtue of the fact that there may be more RAM within the sensing range of the scintillation sensor on one patient relative to another. If, however, the effective sensing range could be limited to a volume falling at or within the vessel volume available in a patient having the smallest blood vessel (i.e. RAM in portions of a larger vessel in a larger patient that is outside the area that the smallest vessel would occupy is not included), then a normalized sensing volume could be utilized across the spectrum of patients, and a more accurate and comparable concentration measurement could be made.

For example, in embodiments where it may be advantageous to measure the concentration of RAM in a blood vessel, it may be desirable to use a probe 100 or cannula 400 designed to have an effective measuring volume approximately equal to the diameter of the smallest blood vessel in which the measurement may be taken (e.g., approx. 5 mm, though other diameters could be used). Accordingly, it may be possible to measure the same volume of space containing RAM (e.g., blood flowing in a blood vessel) in a patient having a smaller blood vessel diameter (e.g., approximately 5 mm) and a patient having a larger blood vessel (e.g., approximately 10 mm). By eliminating, for example, the volume of blood in the larger vessel that lies outside of the exemplary 5 mm effective measurement volume, a more standardized concentration measurement may be taken across a sampling of differently sized patients. Note that other effective volumes may be utilized, including for example vessels approximately 1 mm in diameter to larger vessels that are as much as 20 mm or more in diameter.

Advantageously for purposes of the present disclosure, and as known by those having skill in the art, the distance from which a particle can be detected by scintillation material (e.g., scintillation material 110) is related to: (1) the energy or velocity of the particle when it is expelled from the RAM (for which, the maximum is known in the art for a given RAM); and (2) the rate at which such a particle gives up kinetic energy and decreases in velocity through collisions with other materials in the region (which is also known for a given RAM). Such collision materials may include, for example, water molecules, other materials in the blood travelling through the vessel, and importantly, any other particle absorption materials between the scintillator material 110 and the exterior of the scintillator probe (e.g., light shielding 180 (which may, in some embodiments, extend beyond the areas pictured in the Figures) or other particle absorption materials (discussed further hereinbelow)). Thus, a measurement of the kinetic energy of the particle when interacting with the scintillation material 110 may describe the distance it has traveled since first expelled from RAM. Examples of different types of RAM (i.e., isotopes) that may be used in the body, and their associated energy and known range in water, may include, but are not limited to, the following:

| Isotope | Max Energy (MeV) | Max Range in Water (mm) |
|---|---|---|
| Carbon-14 | 0.156 | 0.3 |
| Sulfur-35 | 0.166 | 0.4 |
| Lutetium-177 | 0.49 | 1.6 |
| Iodine-131 | 0.606 | 2 |
| Fluorine-18 | 0.635 | 2.4 |
| Carbon-11 | 0.961 | 3.9 |
| Nitrogen-13 | 1.19 | 5.1 |
| Phosphorus-32 | 1.709 | 7.6 |
| Oxygen-15 | 1.723 | 8 |
| Gallium-68 | 1.899 | 8.9 |
| Yttrium-90 | 2.281 | 11 |
| Rubidium-82 | 3.35 | 17 |

Accordingly, and referring again to FIG. 4, various embodiments of the cannula 400 may also include one or more particle absorption materials 175, wherein the particle absorption material 175 may be configured to have a first energy blocking threshold. Use of such particle absorption materials 175 may advantageously limit the effective volume from which particles emitted by RAM may be detected. Particle absorption material 175 may include, among other things, light shielding material 180, needle material 130, probe material 159, and/or any other material capable of blocking all or a desired portion of particles having energies below the desired threshold. Examples of other suitable particle absorption materials may include, but are not limited to, one or more of aluminum, titanium, nitinol (nickel-titanium), gold, silver, cobalt-chrome, stainless steel, PMMA (poly(methyl methacrylate)), PVC (polyvinyl chloride), polyethylene, PEEK (polyether ether ketone), Polycarbonate, PEI (polyetherimede), polysulfone, polypropylene, polyurethane, and the like.

Additionally, in some embodiments, it may be advantageous to incorporate particle absorption material 175′ having a second energy blocking threshold that may be positioned, for example, substantially between the delivery lumen 410 and the scintillation material 110 to, for example, block unwanted particles emitted from residual RAM remaining in delivery lumen 410 following an injection of RAM into the body. Particle absorption material 175′ may be the same as particle absorption material 175 (and/or have a second energy blocking threshold substantially equal to the first energy blocking threshold), or particle absorption material 175′ may be distinguishable from particle absorption material 175, and have a second energy blocking threshold distinguishable from the first energy blocking threshold of particle absorption material 175.

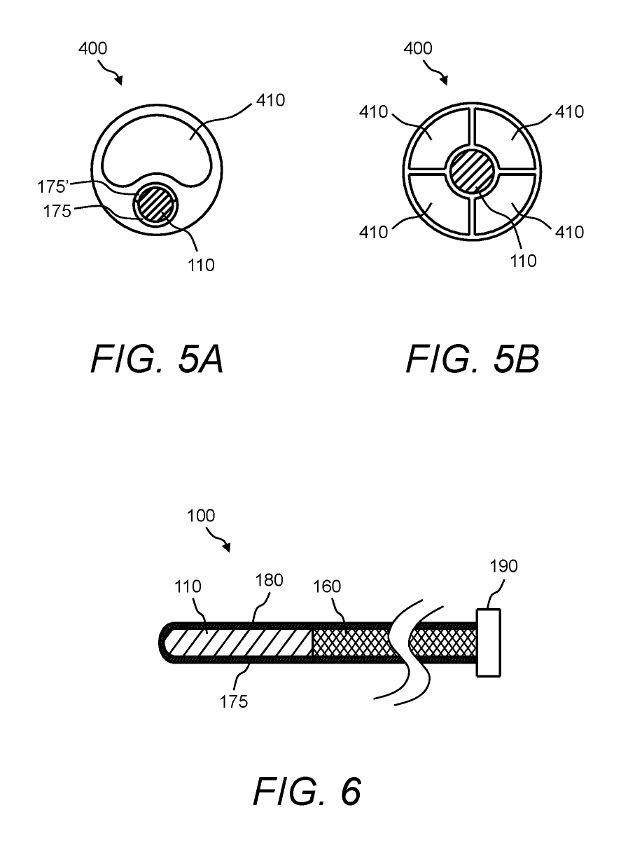

Referring now to FIG. 5A, an exemplary cross-sectional view of cannula 400 illustrated for example in FIG. 4 is presented wherein the delivery lumen 410 may include a single lumen extending substantially adjacent a section of scintillation material 110. Once again, cannula 400 may include, as desired, one or more layers of particle absorption materials 175 (or 175′) to block detection of one or more of residual RAM stuck in delivery lumen 410, and/or reduce the effective measuring volume of the device generally.

Referring now to FIG. 5B, an alternative embodiment of cannula 400 is presented that may include, among other things, more than one separate delivery lumen 410. In some embodiments, the two or more delivery lumens 410 substantially surround scintillation material 110.

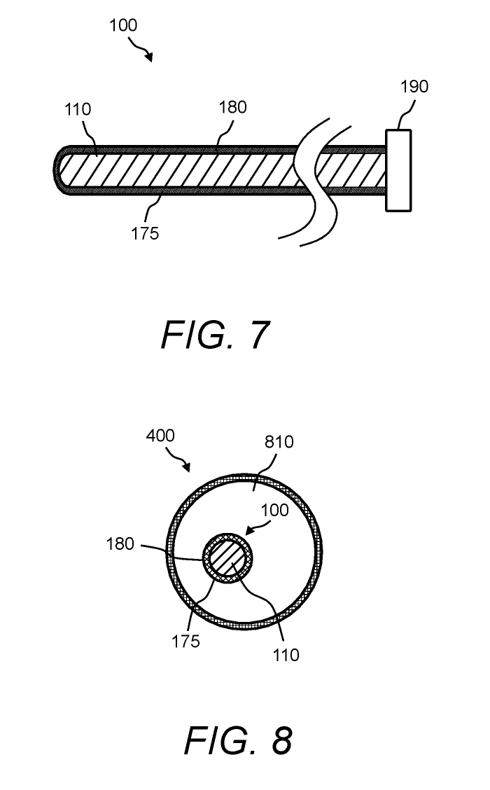

Referring now to FIG. 6, FIG. 7, and FIG. 8, various embodiments of the present disclosure are presented wherein a scintillation assembly can be placed through a catheter or existing lumen. In FIG. 6, an example of a scintillation probe 100 comprising scintillation material 110, and/or fiber optic material 160 is presented, wherein all or a portion of the scintillation material 110 may be covered in light shielding material 180. In some embodiments, light shielding material 180 may be, or may also include, particle absorption material 175. In some additional embodiments, the scintillation material 110 in combination with fiber optic material 160 can be of sufficient length to protrude into, for example, a patient's vasculature as well as exit the patient and attach to an external connector 190. FIG. 7 presents another embodiment of probe 100 wherein the scintillation material 110 may extend within the light shielding material 180 (and/or particle absorption material 175) to an optical connector 190 that may be positioned outside a patient's body, thereby obviating the need for any fiber optic material.

Referring now to FIG. 8, a longitudinal cross sectional view of an exemplary cannula 400 having delivery lumen 810 (e.g., a catheter) is presented, wherein probe 100 may be inserted within, or otherwise incorporated into, the lumen 810. Such embodiments could, for example, allow for simultaneous sensing and injecting, among other things. For example, RAM could be injected into the patient via delivery lumen 810, and the level of RAM in a volume to be measured within the patient could be measured using, for example, probe 100 having scintillation material 110 and, if desired, light absorbing material 180 and/or particle absorption material 175.

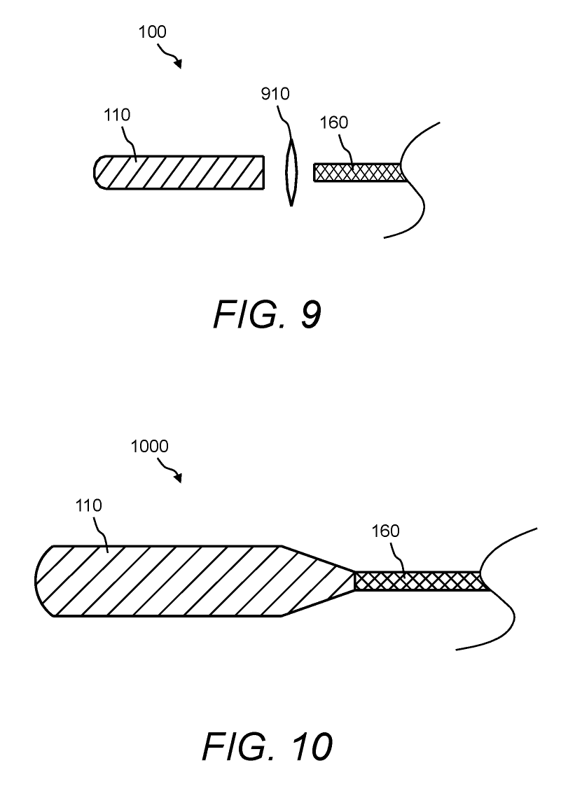

Various embodiments of probe 100 (or cannula 400) may also make use of one or more lenses such as, for example, lens 910 presented in FIG. 9. Such lenses may, for example, focus light generated within scintillation material (e.g., scintillation material 110) onto fiber optic material (e.g., fiber optic material 160) for transmission to an optical connector or optical sensor (e.g., optical connector 190).

Light may also be focused to the end of a transmission fiber (e.g., fiber optic material 160) by way of, for example, shaping or grinding the scintillation material. Referring now to FIG. 10, exemplary shaped scintillation material 1000 that is shaped in such a manner is presented. Such shaping and/or grinding may be useful, for example, when transitioning between differing diameters of scintillation material 110 and fiber optic material 160, and other scenarios.

In some embodiments, one or more optical detectors for detecting light emitted from scintillation material can be utilized for converting the light signals into electrical signals that may be processed by, for example, a computer or other device, rather than such a device interpreting the optical signal directly. The placement of such optical detectors can vary, and may include for example placement both inside and outside of the vessel containing the fluid to be measured. The electrical signals generated by such optical detectors may also be transmitted using any other appropriate means.

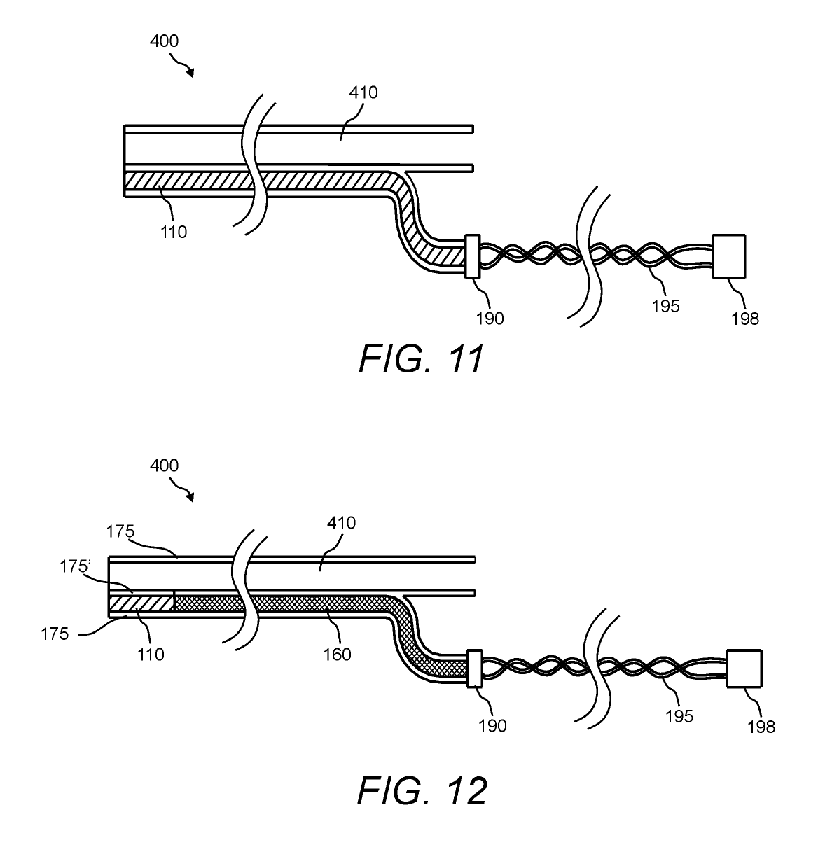

Referring now to FIG. 11, another exemplary embodiment of cannula 400 having delivery lumen 410 is presented wherein an optical detector 190 is in direct contact with scintillator material 110. Such a detector may then convert the optical signal from the scintillator material 110 into an electrical signal that may be transmitted using any appropriate means, including for example, a twisted pair of electrical cabling 195, or other means known in the art (e.g., coaxial cabling, Ethernet, etc.). The electrical cabling 195 may then be coupled to an electrical connector 198 that can then interface as appropriate with a computer or other user interface. Data from detector 190 may also be communicated to a computer other device using any appropriate wireless means, including, for example, Bluetooth, RF, Wi-Fi, etc.

Alternatively, and referring now to FIG. 12, an alternative embodiment of the device illustrated in FIG. 11 is presented, wherein a length of fiber optic material (e.g. fiber optic material 160) may be utilized. In such embodiments, the fiber optic material may be coupled to an optical detector 190 and then transmitted electrically as previously disclosed (e.g., via electrical cabling 195 to electrical connector 198). Cannula 400 of FIG. 12 may also include, as discussed hereinabove, a first layer of particle absorption material 175′ having a first energy blocking threshold may be included between the delivery cannula 410 and the scintillation material 110 to, for example, block unwanted particles emitted from RAM remaining in the delivery cannula following injection of RAM into a patient. Further, additional particle absorption materials 175 may be included substantially about the scintillation material 110 and/or the cannula 400 as a whole to block unwanted particles emitted from RAM falling outside of a desired measurement volume (e.g., a volume lying outside of a vessel to be measured).

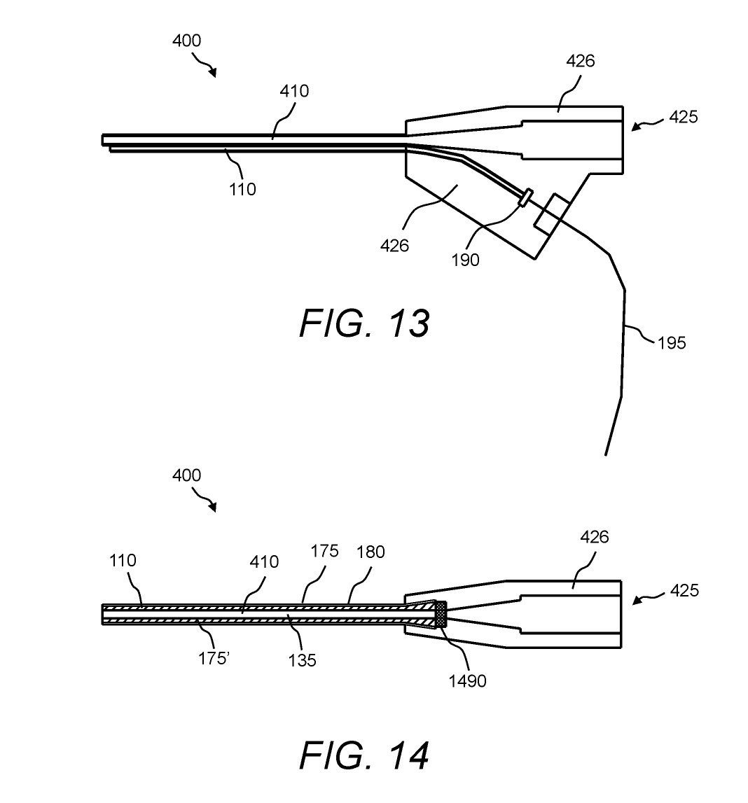

Referring now to FIG. 13, yet another embodiment of the present disclosure is presented wherein a cannula 400 may be integrated with a delivery hub 425 and an embedded optical sensor 190. Cannula 400 in FIG. 13 may include a length of scintillation fiber 110 that may extend substantially adjacent to the delivery lumen 410. The scintillation fiber 110 may be terminated within material 426 surrounding delivery hub 425 and the light signal generated within the scintillation fiber 110 converted to an electrical or other usable signal at sensor 190 as previously disclosed, and optionally transmitted to a separate user interface, computer or other device (via, for example, wire 195). Such embodiments may have the advantage of providing a single catheter unit that may be used to both administer RAM and/or other materials (via, for example, delivery lumen 410) to the vessel and simultaneously detect RAM (via, for example, scintillation fiber 110) in the vessel. Light shielding material (e.g., light shielding material 180) and/or particle absorption material 175 may also be utilized as appropriate to properly shield the scintillation fiber 110 and/or sensor 190 as appropriate and disclosed hereinabove.

Referring now to FIG. 14, another exemplary embodiment of a cannula 400 is presented wherein the delivery lumen 410 may be itself made in whole or in part of scintillation material (e.g. scintillation material 110). Like cannula 400 illustrated in FIG. 13, the cannula 400 in FIG. 14 may include a single catheter unit wherein the delivery lumen 410 is made, in whole or in part, from scintillation material 110 and forms a hollow core 135 (i.e. delivery lumen 410) that may be used to both administer RAM and/or other materials to the vessel (or extract material from the body) and simultaneously detect RAM in the vessel. In this exemplary embodiment, a ring-shaped light detector 1490 may be employed to better engage with the ring-shaped scintillator material 110 of delivery lumen 410. The ring-shaped light detector 1490 may be disposed within a material 426 surrounding a delivery hub 425. The cannula 400 of FIG. 14 may also include light shielding material 180 and/or particle absorption material 175 (and/or 175′) as necessary for preventing light and/or particles from undesirably entering the system or affecting the measurements being taken, as discussed hereinabove.

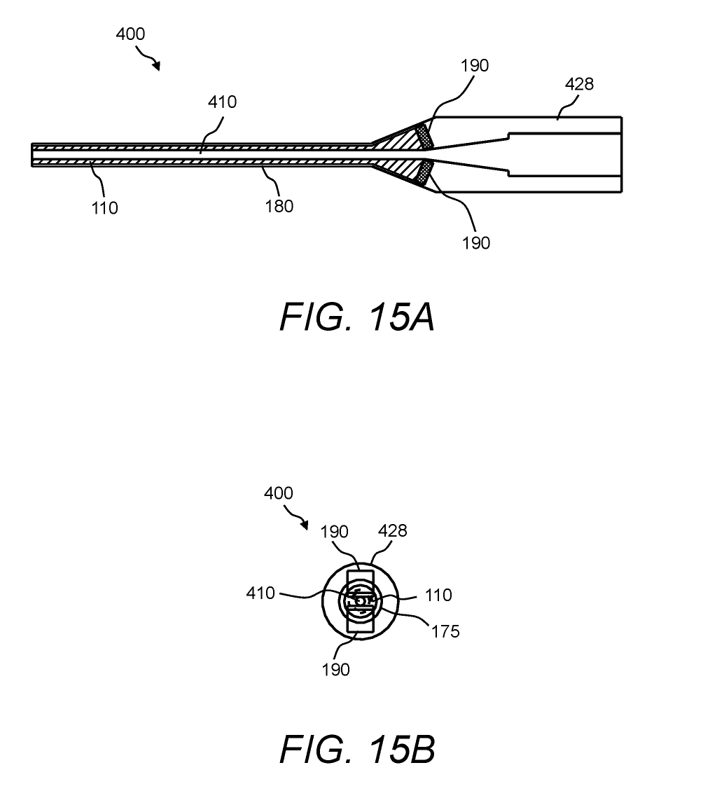

Referring now to FIG. 15A and FIG. 15B, in some embodiments, two or more light detectors 190 may be disposed, for example, at the axial end of the scintillation material 110 that makes up all or part of delivery lumen 410 such that light generated within the scintillation material 110 travels axially along the scintillation material 110 to the two or more light detectors 190. In various other embodiments, three, four, or any other number of light detectors may be disposed at, for example, the axial end of the scintillation material 110. Light detectors 190 may be disposed equidistant about the longitudinal axis of the delivery lumen 410, or disposed in any other appropriate configuration to receive light generated within the scintillation material. In some embodiments, fiber optic material (e.g., fiber optic material 160) may be included within or otherwise adjacent to the scintillation material 110 in delivery lumen 410 such that light generated within the scintillation material 110 travels axially along the delivery lumen 410 from the scintillation material 130 through the fiber optic material 160 and to the light detectors 190. Light shielding (e.g., light shielding 180) may also be included in cannula 400 as desired and otherwise described herein to prevent exposure of unwanted light to the system and/or light detectors 190. Particle absorption material 175 may also be utilized as discussed hereinabove.

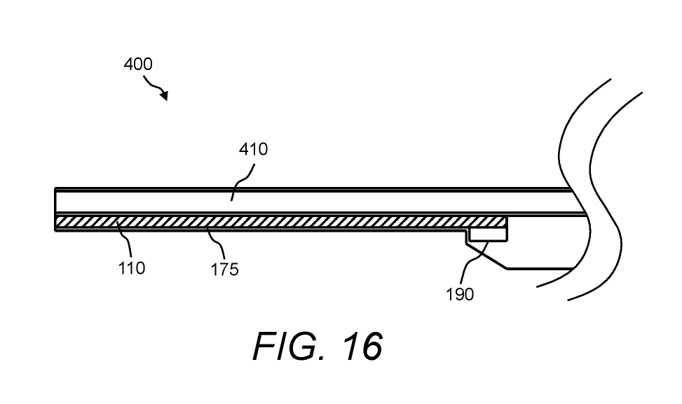

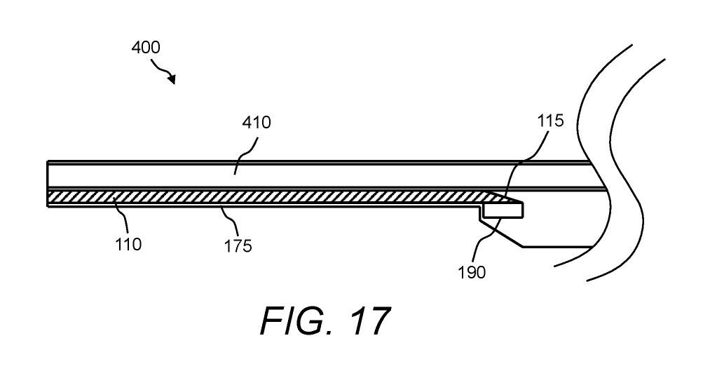

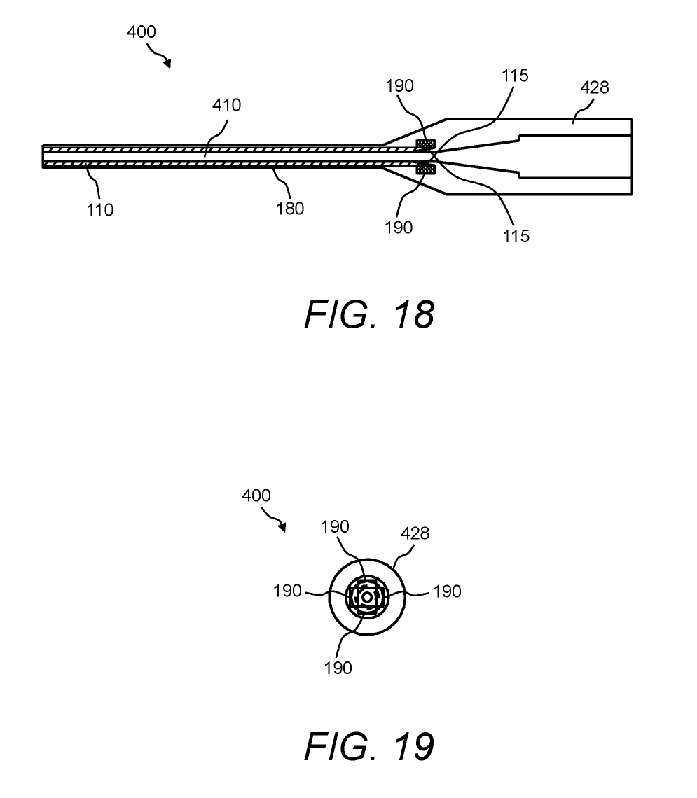

Referring now to FIG. 16, another exemplary embodiment of the presently disclosed cannula 400 is presented wherein scintillation material 110 and light detector 190 may be disposed substantially adjacent a delivery lumen (e.g., delivery lumen 410). In some embodiments, light detector 190 may be disposed radially adjacent to the scintillation material 110 rather than, for example, at the axial end of the scintillation material as shown, for example, in FIG. 15A and FIG. 15B. Particle absorption material 175 may also be utilized as discussed hereinabove. Referring now to FIG. 17, scintillation material 110 may in some embodiments be formed in a manner to create a surface 115 that may be used to, among other things, reflect light traveling substantially along a longitudinal axis of the scintillation material 110 and redirect such light in a substantially radial direction onto one or more light detectors 190. Particle absorption material 175 may also be utilized as discussed hereinabove. FIG. 18 and FIG. 19 include an alternative embodiment of cannula 400 in FIG. 17 that includes a reflective surface(s) 115 and one or more light sensors 190 disposed radially about all or a portion of the scintillation material 110.

In some embodiments, it may also be advantageous to ensure that the probe (e.g. probe 100 or cannula 400) is substantially centered within the vessel to ensure that the effective measurement volume is contained within the vessel. In some embodiments, having the probe 100 or cannula 400 substantially centered may mean, for example, that the effective measurement volume of the probe 100 or cannula 400 falls within the blood vessel of interest.

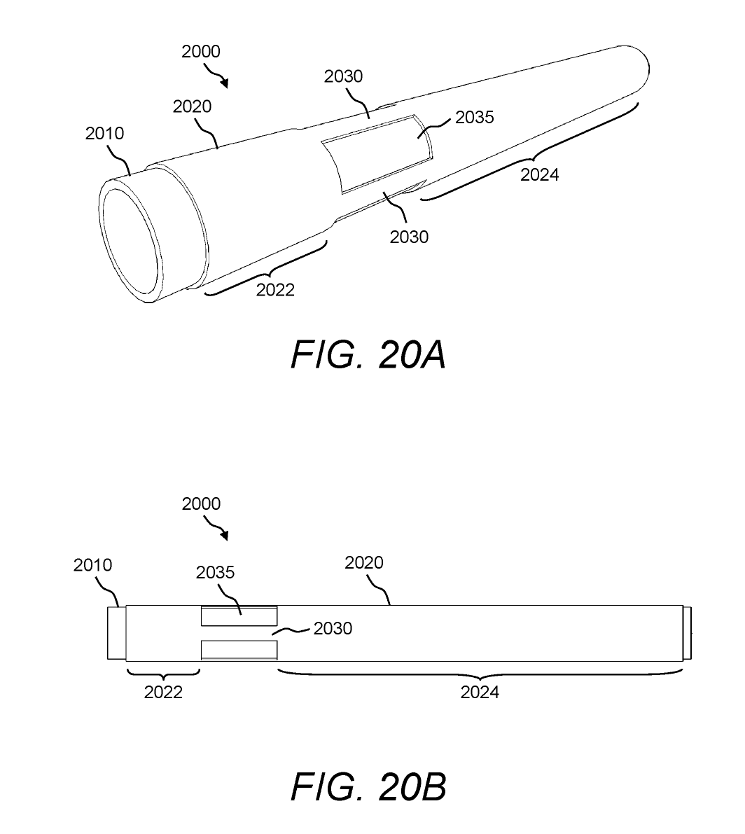

Referring now to FIG. 20A and FIG. 20B, an exemplary embodiment for a mechanism for centering a catheter 2010 within a vessel (e.g., a blood-vessel) is illustrated. In some embodiments, the self-centering catheter system 2000 may be atraumatic and include a catheter 2010 surrounded by a sheath 2020. Sheath 2020 may include, among other things, a first solid portion 2022, a second solid portion 2024, and one or more connecting strips 2030 spaced apart by one or more windows 2035. In some embodiments, sheath 2020 may include four connecting strips 2030, but sheath 2020 may include two, three, five, six, or any other number of strips as desired. In such embodiments, the two or more connecting strips 2030 may each be substantially the same width, or may be of substantially different widths, or any combination thereof as desired. Similarly, windows 2035 may each be substantially the same width, or may be of substantially different widths, or any combination thereof as desired. In various other embodiments, sheath 2020 may also include one solid connecting strip 2030 with no windows 2035, or one connecting strip with one window 2035. In such embodiments, the one connecting strip 2030 may be larger than, smaller than, or the same size as the window 2035. FIG. 20A and FIG. 20B illustrate the self-centering catheter system 2000 in a first “insertion” position according to some embodiments of the present disclosure.

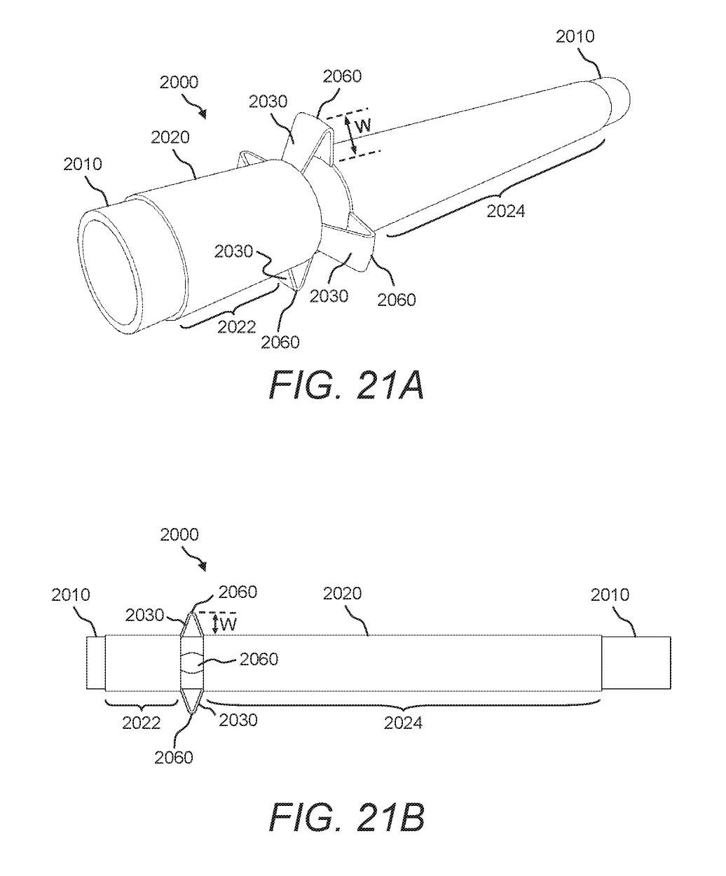

Referring now to FIG. 21A and FIG. 21B, a second “activated” position of self-centering catheter system 2000 is presented according to some embodiments of the present disclosure. For example, in some embodiments, the first solid portion 2022 and second solid portion 2024 may be movable relative to catheter 2010 (e.g., capable of sliding along catheter 2010). In some embodiments, catheter 2010 is a 22-gauge (0.9 mm) catheter, but the catheter may also range as desired from about 14 gauge to 26 gauge, and/or below 14 gauge and above 26 gauge if desired. The second solid portion 2024 may also be operatively movable along catheter 2010 relative to first solid portion 2022 such that second solid portion 2024 may operatively slide along catheter 2010 towards and relatively adjacent to first solid portion 2022 as depicted in, for example, FIG. 21 A and FIG. 21B. In so moving, in some embodiments, connecting strips 2030 may be compressed, causing connecting strips 2030 to bulge outwards as shown, for example, in FIGS. 21A and 21B, thereby forming wings 2060. By varying the length of connecting strips 2030, the effective radial width of the wings “W” can be advantageously varied along with the effective diameter of the catheter 2010 and sheath 2020 to accommodate any desired vessel diameter (i.e., ensure that the overall combined width of the wings “W” do not extend beyond a minimum vessel diameter so as to prevent damage to, among other things, the vessel wall(s)). In some embodiments, the total diameter of the catheter system 2000 with wings 2060 in the activated (i.e. extended) position is approximately 5 mm, but may be less than 5 mm and as large as 10 mm or more if desired. In some embodiments, the radial width W of wings 2060 may be approximately one-half the length of connecting strips 2030. In other embodiments, the radial width W of wings 2060 may be less than one-half of the length of connecting strips 2030.

In some embodiments, sheath 2020 may be configured such that sheath 2020 defaults to the first “insertion” position as depicted in FIG. 20A and FIG. 20B. In such embodiments, connecting strips 2030 may be of a such rigidity that strips 2030 tend to push first solid portion 2022 and second solid portion 2024 apart from one another. In various other embodiments, sheath 2020 may be configured such that sheath 2020 defaults to the second “activated” position as depicted for example in FIG. 21A and FIG. 21B. In such embodiments, connecting strips 2030 may include metal or other rigid material that default to the activated position but extend substantially flat to the first “insertion” position when second solid portion 2024 is made to slide away from first solid portion 2022. It is also contemplated that in some embodiments, first solid portion 2022 and second solid portion 2024 are not entirely solid, but may include any composition suitable for causing the compression or expansion of connecting strips 2030 described hereinabove. For example, first solid portion 2022 and/or second solid portion 2024 may include strips of material, a lattice structure, or other structurally suitable configuration. Sheath 2020 may be made from any suitable material, including among other things biocompatible metals, plastics, and the like.

According to some embodiments, the present disclosure also provides for a method of using scintillation probe disclosed hereinabove. In some embodiments, a scintillation probe as taught herein may be inserted to a patient's blood vessel. In some embodiments, a mechanism (e.g., sheath 2020) may be utilized to substantially center the probe in the vessel. The probe may then measure the presence of, and/or the level of, RAM in the blood contained within the vessel in real time. Various means for capturing and displaying the presence or levels of RAM in the blood may be utilized, including those taught in U.S. Pat. No. 9,002,438 and U.S. Patent Publication No. 2015/0276937, both of which are incorporated herein by reference in their entireties.

The present disclosure further contemplates use of various embodiments in industrial settings. For example, variations of the present disclosure could be used to measure RAM in any fluid carried within any fluid carrying vessel. For example, RAM levels could be measured in oil pipelines for use in detecting the presence of leaks or other flow issues. While examples of use in relation to blood vessels is discussed in detail above, the inventors do not intend such disclosure to be limiting and expressly contemplate use of scintillation materials in any type of fluid-carrying vessels for measuring the presence of or level of RAM in a fluid carried therein.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments +50%, in some embodiments ±20%, in some embodiments +10%, in some embodiments ±5%, in some embodiments +1%, in some embodiments ±0.5%, and in some embodiments +0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A scintillation device for localized measurement of radiotracers in a blood vessel of interest, the device comprising:
a cannula comprising scintillation material and a delivery lumen;
wherein the scintillation material emits light when impacted with particles emitted from a radioactive material, and at least a portion of the light is received by an optical connector;
and further wherein the cannula is sized to deliver the delivery lumen and the scintillation material to a position inside the blood vessel of interest.

2. The scintillation device of claim 1, wherein the cannula further comprises a fiber optic material.

3. The scintillation device of claim 2, wherein the at least a portion of the light propagates via the fiber optic material to optical connector.

4. The scintillation device of claim 2 wherein the scintillation material is shaped to focus light to at least one of the fiber optic material or optical connector.

5. The scintillation device of claim 1 wherein the optical connector is an optical detector, and further wherein the optical detector converts the received at least a portion of the light into an electrical signal for processing.

6. The scintillation device of claim 1 wherein the cannula comprises needle material.

7. The scintillation device of claim 1 wherein the delivery lumen comprises two or more separate delivery lumens.

8. The scintillation device of claim 1 further comprising one or more lenses.

9. The scintillation device of claim 1 further comprising one or more wings for substantially centering the scintillation device within the blood vessel of interest.

10. The scintillation device of claim 9, wherein the one or more wings are operatively movable from a first retracted position to a second extended position, thereby permitting insertion of the scintillation device into the blood vessel of interest with the one or more wings in the first retracted position and subsequently taking measurements with the scintillation device in the blood vessel of interest with the one or more wings in the second extended position.

11. The scintillation device of claim 1 further comprising a particle absorption material substantially surrounding the scintillation material, wherein the particle absorption material comprises an energy blocking threshold corresponding to its absorption of energy from the particles emitted from the radioactive material, and further wherein the particle absorption material comprises a thickness configured to effectively block particles having an energy at the particle absorption material below a desired threshold, thereby reducing the effective volume from which the emitted particles are measured.

12. The scintillation device of claim 11 wherein the particle absorption material comprises at least one of PEEK and gold.

13. The scintillation device of claim 11 wherein the particle absorption material substantially between the delivery lumen and the scintillation material comprises a first energy blocking threshold, and the remainder of the particle absorption material comprises a second energy blocking threshold.

14. A scintillation device for localized measurement of radiotracers in the body, the device comprising:
a delivery lumen and scintillation material, wherein the delivery lumen is coupled to a delivery hub, and the scintillation material is optically coupled to a light detector;
and further wherein the delivery lumen and scintillation material are sized to be positioned at least partially within a blood vessel.

15. The device of claim 14 wherein the light detector is housed within the device, and further comprises a signal port for transmitting a signal to an external reader.

16. The device of claim 15 wherein the scintillation material axially surrounds the delivery lumen.

17. The device of claim 16 further comprising a first area of particle absorption material between the delivery lumen and the scintillation material, wherein the first particle absorption material comprises a first energy blocking threshold.

18. The device of claim 17 further comprising a second area of particle absorption material axially surrounding the scintillation material, wherein the second area of particle absorption material comprises a second energy blocking threshold.

19. The device of claim 14 wherein further comprising one or more wings for substantially centering the device within the blood vessel.

20. A system for measuring concentration of radioactive material in the body, the system comprising:
a cannula comprising scintillation material and a delivery lumen, wherein the scintillation material emits light when impacted with particles emitted from a radioactive material, and at least a portion of the light is received by an optical connector, and further wherein the cannula is sized to deliver the delivery lumen and the scintillation material to a position inside the blood vessel of interest; and
a processing system in operable communication with the optical connector, and further wherein the processing system is configured to processes the signals from the optical connector and compute a radioactive material concentration measurement.
